(19) United States
(12) Patent Application Publication
(10) Pub. No.: US 2025/0177347 A1
(43) Pub. Date: Jun. 5, 2025

(54) SCLAREOL OR SCLAREOLIDE FOR USE IN THE TREATMENT OF INFLAMMATORY SKIN CONDITIONS

(71) Applicant: ISP Investments LLC, Wilmington, DE (US)

(72) Inventors: Imane GARCIA, Nice (FR); Christophe CAPALLERE, Nice (FR); Marianne ARCIONI, Le Bar Sur Loup (FR); Céline MEYRIGNAC, Puget sur Argens (FR)

(73) Assignee: ISP Investments LLC, Wilmington, DE (US)

(21) Appl. No.: 18/843,215

(22) PCT Filed: Mar. 1, 2023
(86) PCT No.: PCT/US23/14226

§ 371 Date: Aug. 30, 2024

Related U.S. Application Data
(60) Provisional application No. 63/333,959, filed on Apr. 22, 2022.
(60) Provisional application No. 63/315,502, filed on Mar. 1, 2022.

(51) Int. Cl. A61K 31/365; A61K 9/00; A61K 31/047; A61P 17/00; A61P 31/10

(52) U.S. Cl. CPC A61K 31/365; A61K 9/0014; A61K 31/047; A61P 17/00; A61P 31/10

(57) ABSTRACT

The present disclosure relates to the field of personal care and more particularly to a method for preventing or treating inflammatory skin conditions including atopic dermatitis and seborrheic dermatitis which comprises applying to the skin a composition comprising an active agent selected among sclareol, sclareol-like, sclareolide or sclareolide-like and a physiologically acceptable carrier.

Another object of the present invention is a method for improving the aspect of inflammatory prone skin, which comprises applying to the skin a composition comprising an active agent selected among sclareol, sclareol-like, sclareolide or sclareolide-like and a physiologically acceptable carrier.

### FIELD OF THE INVENTION

The present disclosure relates to the field of personal care and more particularly to a method for improving the aspect of inflammatory prone skins, which comprises applying to the skin a composition comprising an active agent selected among sclareol, sclareol-like, sclareolide or sclareolide-like and a physiologically acceptable carrier.

The present disclosure also relates to the field of dermatology and more particularly to a method for the treatment of inflammatory skin conditions including atopic dermatitis or seborrheic dermatitis which comprises applying to the skin a composition comprising an active agent selected among sclareol, sclareol-like, sclareolide or sclareolide-like and a physiologically acceptable carrier.

### BACKGROUND

Inflammatory skin conditions, also known as dermatitis, are a common group of skin disorders, characterized by skin inflammation and redness. In the word “dermatitis,” “derm” means “skin” and “itis” means “inflammation.”. There are several types of dermatitis, including irritant contact dermatitis, allergic contact dermatitis, atopic dermatitis, psoriasis and seborrheic dermatitis, among others.

Atopic dermatitis is thought to result from a combination of genetic predisposition and environmental triggers. Inflammation is a key characteristic of atopic dermatitis and is believed to play a crucial role in its development. The skin of individuals with atopic dermatitis is thought to be more susceptible to injury, leading to a cycle of itching, scratching, and further inflammation (Guttman-Yassky E, Waldman A, Ahluwalia J, Ong P Y, Eichenfield L F. Atopic dermatitis: pathogenesis. Semin Cutan Med Surg. 2017;36 (3): 100-103). This chronic inflammation can result in damage to the skin barrier, which can allow for the entry of irritants and allergens, thereby worsening symptoms of atopic dermatitis.

Seborrheic dermatitis is a condition characterized by scaly, red patches on the scalp, face, and other oily areas of the body. Although the exact causes of seborrheic dermatitis are not well understood, several factors have been implicated, including genetic predisposition, hormonal imbalances, and the presence of specific microorganisms on the skin. Among these microorganisms, Malassezia yeasts have been shown to play a key role in the development of seborrheic dermatitis (Gaitanis G, Magiatis P, Hantschke M, Bassukas I D, Velegraki A. The Malassezia genus in skin and systemic diseases. Clin Microbiol Rev. 2012; 25 (1): 106-141).

Malassezia yeasts designates a lipophilic type of yeasts that are normal resident of healthy skin, including the scalp and face and are thought to play a role in skin hydration and barrier function. They feed on sebum, the oily substance produced by sebaceous glands, and are an important part of the skin's natural microflora (Borda L J, Wikramanayake T C. Seborrheic Dermatitis and Dandruff: A Comprehensive Review. J Clin Investig Dermatol. 2015;3 (2): 10.13188/2373-1044.1000019).

The overgrowth of Malassezia yeast on the skin can lead to the production of by-products, including free fatty acids and indoles, that have been implicated in stratum corneum damage (Wikramanayake T C, Borda L J, Miteva M, Paus R. Seborrheic dermatitis-Looking beyond Malassezia. Exp Dermatol. 2019; 28 (9): 991-1001).

There remains a need for novel natural active agents free of side effects for the treatment of inflammatory skin conditions including for example, atopic dermatitis and seborrheic dermatitis or for improving the aspect of inflammatory prone skins.

The objective of the present invention is to provide a skin active agent which is non-irritant to the skin, while at the same time effectively improves skin condition.

Another object of the present invention is to provide a natural anti-inflammatory agent with an antifungal effect against Malassezia species and more precisely against Malassezia restricta.

The anti-inflammatory activity of sclareol and sclareolide is described in PCT Publication WO2002/030385. The anti-microbial activity of sclareol-like and sclareolide-like compounds is already described in U.S. Pat. No. 6,150,381. In PCT Publication WO 2001/074327, the use of sclareolide as cell differentiation enhancer is disclosed. According to this patent differentiation enhancers like sclareolide are used to stimulate the production of lipids from epidermal cells, and concurrently increase the lipid content of the barrier. The strengthening of barrier function of the skin using sclareolide is described in PCT Publication WO 2002/060381 A2. The use of sclareolide in cosmetic formulations used to enhance the stratum corneum function is described in U.S. Publication 2010/0247692.

Sclareolide is endowed with a good antibacterial activity against Staphylococcus aureus ATCC 25923, Pseudomonas aeruginosa ATCC 27950, Escherichia coli ATCC 25922 and Enterococcus faecalis ATCC 29212 (Hayet E, et al. Antibacterial and cytotoxic activity of the acetone extract of the flowers of Salvia sclarea and some natural products. Pak J Pharm Sci. 2007 April;20 (2): 146-8).

Sclareol have been shown to inhibiting growth, biofilm formation, and yeast-hyphal transition of several fungi strains including C. albicans KCTC7965, C. tropicalis var. tropicalis KCTC17762, C. parapsilosis var. parapsilosis KACC45480, C. parapsilosis KACC49573, C. glabrata KCTC7219, and C. auris KCTC17809 (Kim C, Kim J-G, Kim K-Y. Anti-Candida Potential of Sclareol in Inhibiting Growth, Biofilm Formation, and Yeast-Hyphal Transition. Journal of Fungi. 2023; 9 (1): 98)

For all the above reasons, sclareol and sclareolide were identified as potential candidates to treat inflammatory skin disorders. The applicant has now found that the use of a composition comprising sclareol, sclareol-like, sclareolide or sclareolide-like as an active agent makes it possible to inhibit Malassezia restricta growth and its consequences.

Ketoconazole is the conventional antifungal molecule used for seborrheic dermatitis and dandruff treatment. Recently, beyond his antifungal effect on Malassezia, it was argued that ketoconazole shifts host lipid profile from skin which affects Malassezia lipid metabolism (Goularte-Silva V, Paulino L C. Ketoconazole beyond antifungal activity: Bioinformatics-based hypothesis on lipid metabolism in dandruff and seborrheic dermatitis. Exp Dermatol. 2022;31 (5): 821-822).

Vitamin D plays an important role in skin structure wherein it stimulates differentiation of keratinocytes to form the upper layers of the epidermis (Bikle D. Vitamin D Metabolism and Function in the Skin. Mol Cell Endocrinol. 2011; 347 (1-2): 80-89). The active form of vitamin D (1,25(OH)2D3) regulates many physiological processes in the skin ranging from cellular proliferation, differentiation, and apoptosis to barrier maintenance and immune functions. Vitamin D deficiency is associated with the risk of psoriasis and atopic dermatitis, and several clinical/observational studies have suggested the beneficial effect of vitamin D in the therapy of these 2 inflammatory skin disorders (Umar M. et al. Vitamin D and the Pathophysiology of Inflammatory Skin Diseases. Skin Pharmacol Physiol 2018; 31, 74-86). The enzyme CYP27B1 metabolizes the intermediary form of vitamin D (25 OH D3) into the active form of vitamin D (1,25(OH)2D3)

### SUMMARY

Disclosed herein is a method for improving the aspect of inflammatory prone skin which comprises applying to the skin a composition comprising an active agent selected among sclareol, sclareol-like, sclareolide or sclareolide-like and a physiologically acceptable carrier.

Also disclosed herein is a method for preventing or treating inflammatory skin conditions including atopic dermatitis or seborrheic dermatitis which comprises applying to the skin a composition comprising as an active agent sclareol, sclareol-like, sclareolide or sclareolide-like and a physiologically acceptable carrier.

### BRIEF DESCRIPTION OF THE DRAWINGS

For the purpose of illustrating aspects of the present invention, there are depicted in the drawing's certain embodiments of the invention. However, the invention is not limited to the precise arrangements and instrumentalities of the embodiments depicted in the drawings. Further, if provided, like reference numerals contained in the drawings are meant to identify similar or identical elements.

FIG. 1 MIC (Minimum Inhibitory Concentration) determination of sclareolide on Malassezia restricta.

FIG. 2 Sclareolide prevents the disruption of the barrier function and inflammation induced by Malassezia restricta on reconstructed human scalp epidermis.

FIG. 3 Sclareolide improves barrier function through keratin 10 increase.

FIG. 4 Sclareolide improves barrier function through epidermal lipid increase.

### DETAILED DESCRIPTION

Detailed embodiments of the present invention are disclosed herein; however, it is to be understood that the disclosed embodiments are merely illustrative of the invention that may be embodied in various forms. Therefore, specific structural and functional details disclosed herein are not to be interpreted as limiting, but merely as a representative basis for teaching one skilled in the art to variously employ the present invention.

Whenever a term is identified by reference to a range, the range will be understood to explicitly disclose every element thereof. As a non-limiting example, a range of 1-10% will be understood to include 1%, 2%, 3%, 4%, 5%, 6%, 7%, 8%, 9%, and 10%, and all values between 1 and 10%.

Where two or more substituents are referred to as being “selected from” a group of enumerated alternatives, it is meant that each substituent can be any element of that group, independent of the identity of the other substituents.

As used herein, “% refers to % by weight, that is the weight percent of a component in relation to the total weight of the skin care composition (i.e., including any carriers, vehicles, solvents, fillers, or other components added before application to the skin) unless otherwise provided.

All terms used herein are intended to have their ordinary meaning unless otherwise provided. For the purposes of describing and claiming the present invention, the following terms are defined:

The compositions described and used in the present disclosure can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “Application” as used in reference to a hair care composition means to apply or spread the compositions of the present invention onto a human skin surface such as the epidermis.

“Physiologically acceptable” as used herein means, in the skin care compositions of the invention, a medium suitable for use in contact with human skin tissue without undue toxicity, incompatibility, instability, allergic response and the like, capable of being applied to the skin, integuments or lips of the face or the body of mammals or human beings.

“Effective amount” as used herein means an amount of an agent sufficient to significantly induce a positive appearance and/or feel benefit, but low enough to avoid serious side effects (i.e., to provide a reasonable benefit to risk ratio, within the scope of sound judgment of the skilled artisan).

“Inflammatory or atopic or seborrheic dermatitis skin” as used herein means a condition that causes dry, itchy and inflamed skin which in the cases of seborrheic dermatitis mainly affects regions rich in sebaceous glands, such as the scalp, retro-auricular area, face (nasolabial folds, upper lip, eyelids and eyebrows), and the upper chest.

“Inflammatory prone skin” as used herein means a non-pathological condition that causes dryness and redness to the skin.

Subjects to be treated by the methods of the present invention are typically human subjects, although the methods of the present invention may be useful with any suitable subjects known to those skilled in the art, and particularly mammalian subjects including, in addition to humans, horses, cows, dogs, rabbits, fowl, sheep, and the like, for veterinary purposes.

As disclosed herein, the “active agents of the present invention” or “sclareol and/or a derivative” include sclareol, sclareol-like, sclareolide and sclareolide-like compounds.

The present disclosure relates to a method for preventing and treating inflammatory skin disorders comprising applying to the skin a composition comprising an active agent selected among sclareol, sclareol-like, sclareolide or sclareolide-like and a physiologically acceptable carrier.

Sclareol-like agents are diterpene compounds, and include, for example, sclareol, 13-episclareol, ferruginol, salvipisone, aethopisome, neoclerodane, sagequinone, romulogarzone, ortho-benzoquinone, para-benzoquinone, and clariol. Other sclareol-like compounds include abietane and icetexane diterpenoids, languidulane diterpene, paryin and pimarine diterpenes, methylene quinone diterpenoids, manoyl norditerpenoids, multicaulin, salvipimarone and pimarane diterpenoid. See, for example, the types of compounds set forth in Gonzalez et al., Can. J. Chem. 67 (2), 208-212 (1989); Eanthorpe et al., Phytochem. 29, 2145-2148 (1990); Kouzi et al., Helv. Chim. Acta. 73 (8), 2157-2164 1990); Abraham, Phytochem. 36 (6) 1421-1424 (1994); Ulubelen et al. Phytochem. 36 (4), 971-974 (1994); Hanson, Nat. Prod. Rep., 13, 59-71 (1996) and Topcu et al., J. Nat. Prod. 59, 734-737 (1996).

Sclareolide-like compounds are fused-ring diterpene compounds that may be derived from sclareol by chemical or biological techniques known to those skilled in the art; and include, for example, sclareolide, ambrox, and wiedenol. See, for example, the types of compounds set forth in Hanson, Nat. Prod. Rep. 13, 59-71 (1996); Chackalamanni et al., Tetrahedron Letters 36, 5315-5318 (1995); Barrero et al., Tetrahedron Letters 35, 2945-2948 (1994); Martres et al. Tetrahedron Letters 34, 801-8084 (1993) and Barrero et al., Tetrahedron 49 (5), 10405-10412 (1993).

The active agents described above are cosmetically or pharmaceutically acceptable analogs, derivatives, or salts of sclareol or sclareolide. In the practice of the present invention, the active agents may alternatively be substituted with alkyl (both unsaturated and saturated, and branched and unbranched, such as methyl, ethyl, or isopropyl), aryl, halogen, hydroxy, alkoxy, and amino groups, as will be apparent to those skilled in the art.

Any of the active agents of the present invention may be present as an optical isomer, or chiral compound, or as a mixture of optical isomers and chiral compounds.

Sclareol used according to the present disclosure is advantageously obtained by solvent extraction of clary sage. This diterpene is not widely distributed and the most convenient sources are flower heads of clary sage plant. Sclareol is obtained by solvent extraction of clary sage. U.S. Pat. No. 3,060,172 describes a process for the isolation of sclareol from clary sage. See also, U.S. Pat. No. 5,908,771 describes a method for regeneration of salvia species, and U.S. Pat. No. 5,945,546 describes a method for purifying sclareolide, which are incorporated herein in their entirety by reference. Alternatively, sclareol may also be obtained via genetically modified microorganisms such as found in U.S. Pat. Nos. 9,670,494 and 9,745,602.

Sclareolide (CAS Number 564-20-5) used according to the present disclosure is advantageously prepared by either chemical oxidation followed by lactonization of sclareol or by biotransformation of sclareol using a yeast strain. Exemplary methods of producing sclareolide include those methods disclosed in U.S. Pat. No. 5,525,728 to Schneider et al., U.S. Pat. No. 5,247,100 to Gerke et al., and German Patent DE 3942358 to Gerke et al. Briefly, these processes use a ruthenium catalyst and an oxidation step to convert sclareol into a crude sclareolide product.

Other exemplary methods of converting sclareol to sclareolide that are more commonly used include the biotransformation and fermentation methods described in U.S. Pat. Nos. 4,970,163 and 5,212,078, both to Farbood et al. Sclareolide produced by these described methods is normally provided in wet or dry cake form and is generally from about 90% to 95% pure. Furthermore, sclareolide in higher purity can be obtained through crystallization of the wet or dry cake. Sclareolide has also been reported to have therapeutic properties. See, WO 96/20704 to Braquet et al.

The disclosures of these patents setting forth methods of producing sclareolide from sclareol are incorporated herein by reference in their entirety.

In a particular embodiment the active agent of the present disclosure is sclareolide prepared by using flowers of the species Salvia sclarea.

In another particular embodiment the active agent of the present disclosure is the sclareolide in solid form at room temperature and more preferably is sclareolide in the form of a white crystalline solid, provided as a powder.

For example, sclareolide is commercialized by Ashland under the trade Name sclareance™.

The present disclosure provides cosmetic and pharmaceutical formulations comprising the active agents (including the acceptable salts thereof), in physiologically acceptable carriers for oral and topical or transdermal administration.

Advantageously, cosmetic or pharmaceutical formulations are in a form suitable for topical application and may take the form of, for example, a liquid, lotion, aerosol, cream, gel, ointment, rinse off formula or shampoo.

Antifungal effects of the active agents were studied in vitro on selected Malassezia restricta.

When assayed for its Minimal Inhibitory Concentration (MIC), the effectively inhibited the growth of Malassezia restricta. In addition, the sclareolide also prevents the damages to the upper layers of the epidermis induced by Malassezia restricta and thereby decreases the inflammation.

Moreover, sclareol and derivatives can also prevent inflammatory damages to the skin by reinforcing of the skin barrier function.

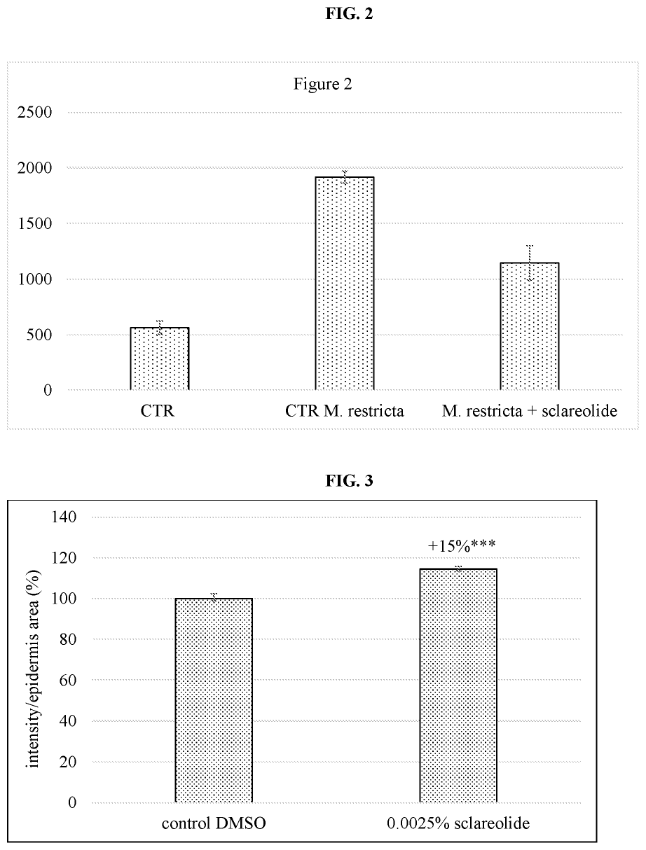

It was shown that sclareolide at 0.12% decreased the inflammation induced by the application of Malassezia restricta on human scalp reconstructed epidermis by the decrease of interleukin-8 release by 40,3% illustrated by FIG. 2.

In a particular embodiment the present disclosure relates to a method for improving the aspect of inflammatory prone skin, which comprises applying to the skin a composition comprising an active agent selected among sclareol, sclareol-like, sclareolide or sclareolide-like and a physiologically acceptable carrier.

In this embodiment the present disclosure relates to a method for decreasing the skin redness which comprises applying to the skin a composition comprising an active agent selected among sclareol, sclareol-like, sclareolide or sclareolide-like and a physiologically acceptable carrier.

In another particular embodiment the present disclosure relates to a method for improving inflammatory skin conditions which comprises applying to the skin a composition comprising an active agent selected among sclareol, sclareol-like, sclareolide or sclareolide-like and a physiologically acceptable carrier.

In another particular embodiment the present disclosure relates to a method for preventing or treating inflammatory skin conditions which comprises applying to the skin a composition comprising an active agent selected among sclareol, sclareol-like, sclareolide or sclareolide-like and a physiologically acceptable carrier.

In another particular embodiment the present disclosure relates to a method for preventing or treating inflammatory skin conditions which comprises applying to the skin a composition comprising an active agent selected among sclareol, sclareol-like, sclareolide or sclareolide-like and a physiologically acceptable carrier, wherein inflammatory skin conditions are dermatitis selected among irritant contact dermatitis, allergic contact dermatitis, atopic dermatitis, psoriasis and seborrheic dermatitis.

In a particular embodiment the present disclosure relates to a method for reinforcing skin barrier function, which comprises applying to the skin a composition comprising an active agent selected among sclareol, sclareol-like, sclareolide or sclareolide-like and a physiologically acceptable carrier.

In skin biopsies cultures treated with a composition comprising 0.12% of sclareolide keratin 10 expression and total lipid content increased leading to a reinforcement of skin barrier function.

In one embodiment the active agent is chosen among sclareol, sclareol-like, sclareolide or sclareolide-like in a concentration ranging from 0.001% to 2.0% by weight, relative to the total weight of the composition.

In another embodiment the active agent is chosen among sclareol, sclareol-like, sclareolide or sclareolide-like in a concentration ranging from 0.001% to 0.2% by weight, relative to the total weight of the composition.

In a preferred embodiment the active agent is the sclareolide in a concentration ranging from 0.001% to 0.2% by weight, relative to the total weight of the composition.

In another preferred embodiment the active agent is sclareolide in a concentration of 0.12% by weight, relative to the total weight of the composition.

A composition comprising from 0.015 to 0.12% of sclareolide showed antifungal properties on Malassezia species and more specifically on Malassezia restricta, in MIC (Minimum inhibitory concentration) assay (EUCAST guidelines, with slight modifications).

Specific embodiments of this cosmetic treatment method also result from the above description. Other advantages and features of the invention will be more apparent upon reading the examples provided for illustrative and non-limiting purposes.

### Examples

The following examples are intended to illustrate particular embodiments of the present invention but are by no means intended to limit the scope of the present invention.

### Example 1: Evaluation of Sclareolide Antifungal Potential on Malassezia restricta

Objective: Sclareolide antifungal potential on Malassezia restricta was evaluated by the broth microdilution method.

Protocol: Malassezia restricta was routinely grown on modified Leeman-Notman (mLN) agar. The broth microdilution assay was performed following EUCAST guidelines (www.eucast.org) with slight modifications. Between five and ten colonies were suspended in a modified RPMI 1640 media supplemented with resazurin at a D.O (Density Optical) of 0.1. In a 96 well plate, serial dilutions of sclareolide (0.00023% to 0.12%) from a 24% stock solution in DMSO, and ketoconazole (0.001 to 0.25 μg/ml) solutions were prepared in modified RPMI 1640 culture medium. An equal volume of Malassezia restricta inoculum was added to the test samples at different concentrations. Following 48 h-60 h of incubation at 34° C., resazurin fluorescence was analyzed at 530 nm and 590 nm.

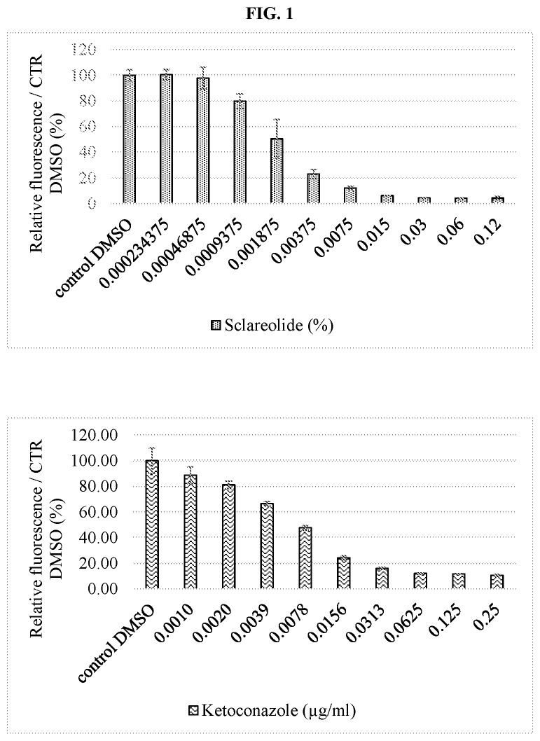

Results and conclusion: Results are presented as relative fluorescence intensity compared to DMSO control. MIC is defined as the lowest drug concentration that prevents growth of microorganisms. Growth of Malassezia restricta was significantly inhibited by sclareolide from the concentration 0.015%, and completely inhibited at the concentration 0.12%, while ketoconazole presented a high inhibition of Malassezia restricta with a MIC of 0.0625 μg/ml as illustrated at FIG. 1.

### Example 2: Sclareolide Prevents the Disruption of the Barrier Function and Inflammation

Objective: Sclareolide anti-inflammatory effect and barrier function prevention was quantified by cytokine quantification and histological observation respectively

Protocol: Malassezia restricta was applied topically to a scalp reconstructed human epidermis. This in vitro model is reconstructed from keratinocytes isolated from scalp skin. More precisely, the location of the epidermis used to isolate keratinocytes is interfollicular epidermis. These interfollicular keratinocytes was harvested and transferred into a cell culture insert and after several days of reconstruction, 100 μl of Malassezia restricta was applied on the top of the epidermis. Two days later, 100 μL sclareolide at a concentration of 0,12% was applied on the top of the epidermis, another application was performed after 24 hours. Ninety-six hours after the beginning of the Malassezia treatment, scalp reconstructed epidermis was stopped to performed histological analysis to observe modifications in the upper layers of the epidermis and the supernatant was harvested to measure inflammatory cytokine release by ELISA method.

Results and conclusion: sclareolide prevented the disruption of the barrier function and the penetration of Malassezia restricta in the upper layers of the epidermis. In parallel, sclareolide decreased the inflammation induced by Malassezia restricta by the decrease of interleukin-8 release by 40,3% illustrated by FIG. 2.

### Example 3: Sclareolide Improves Barrier Function Through Keratin 10 Increase

Objective: The aim of this study is to show the effect of sclareolide on the synthesis of keratin 10 in biopsies of healthy abdominal skin.

Method: keratin 10 was assessed by immunohistochemistry with a specific antibody. Biopsies of healthy human abdominal skin in culture were treated with sclareolide diluted at 0.0025% in 0.1% DMSO applied twice a day for 48 hours topically (20 μl/biopsy). Control biopsies received only 0.1% DMSO. The immunostaining was performed using paraffin sections incubated in the presence of anti-keratin 10 antibody (rabbit monoclonal, Abcam). After an hour and a half of incubation followed by rinses, the sections were incubated in the presence of the secondary anti-rabbit antibody coupled with a fluorophore (Alexa Fluor® 488, Invitrogen). The sections were then examined under a fluorescence microscope (Nikon Eclipse Ni-E microscope). Red fluorescence was then observed (using NiS-AR Nikon acquisition software) and quantified by image analysis (Volocity® image analysis software, Improvision).

Results & conclusion: Sclareolide at 0.0025% was able to increase the levels of keratin 10 in healthy ex vivo abdominal skin biopsies, illustrated in FIG. 3. This effect may be associated with a better quality of the skin barrier function.

### Example 4: Sclareolide Improves Barrier Function Through Epidermal Lipid Increase

Objective: The aim of this study is to show the effect of sclareolide on the synthesis of epidermal lipids in biopsies of healthy abdominal skin.

Method: Epidermal lipids were assessed by fluorescent staining by using the fluorescent dye Nile Red.

Biopsies of healthy human abdominal skin in culture were treated with sclareolide diluted at 0.0025% in 0.1% DMSO applied twice a day for 48 hours topically (20 μl/biopsy). Control biopsies received only 0.1% DMSO. The epidermal lipids staining was performed using paraffin sections incubated with 100 nM of Nile Red solution (Sigma) for 10 minutes. The sections were then examined under a fluorescence microscope (Nikon Eclipse Ni-E microscope). Red fluorescence was then observed (using NiS-AR Nikon acquisition software) and quantified by image analysis (Volocity® image analysis software, Improvision).

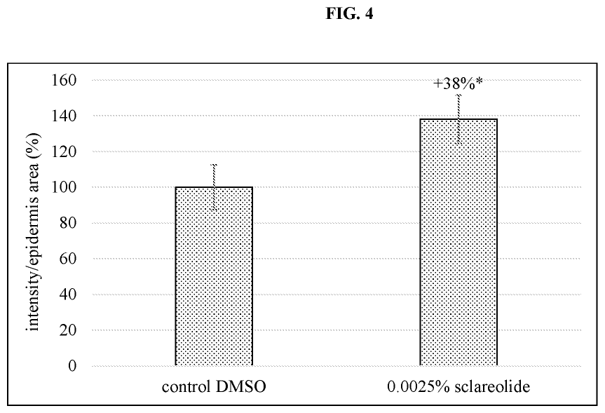

Results & conclusion: Sclareolide at 0.0025% was able to increase the levels of epidermal lipids in healthy ex vivo abdominal skin biopsies, illustrated in FIG. 4. This effect may be associated with a better quality of the skin barrier function.

1. A method for improving the aspect of inflammatory prone skin which comprises applying to the skin a composition comprising an active agent selected among sclareol, sclareol-like, sclareolide or sclareolide-like and a physiologically acceptable carrier.

2. The method of claim 1 wherein the active agent is the sclareolide in a concentration ranging from 0.001% to 0.2% by weight, relative to the total weight of the composition.

3. The method of claim 1 wherein the active agent decreases the growth of Malassezia species and preferably the growth of Malassezia restricta.

4. The method of claim 1 wherein the active agent reduces the redness of the skin.

5. The method of claim 1 wherein the active agent improves skin barrier function.

6. The method of claim 1 wherein in skin cells keratin 10 expression and total lipid content are increased and interleukin-8 expression is decreased.

7. The method of claim 1 wherein the composition is in a form suitable for topical application.

8. The method of claim 1 wherein the composition a is in the form of a liquid, a lotion, an aerosol, a cream, a gel or an ointment.

9. A method for preventing or treating inflammatory skin conditions which comprises applying to the skin a composition comprising an active agent selected among sclareol, sclareol-like, sclareolide or sclareolide-like and a physiologically acceptable carrier.

10. The method of claim 9 wherein the active agent is the sclareolide in a concentration ranging from 0.001% to 0.2% by weight, relative to the total weight of the composition.

11. The method of claim 9 wherein the active agent decreases the growth of Malassezia species and preferably the growth of Malassezia restricta.

12. The method of claim 9 wherein inflammatory skin conditions are dermatitis selected among irritant contact dermatitis, allergic contact dermatitis, atopic dermatitis, psoriasis and seborrheic dermatitis.

13. The method of claim 9 wherein the active agent reduces the redness of the skin and improves skin barrier function.

14. The method of claim 9 wherein in skin cells keratin 10 expression and total lipid content are increased and interleukin-8 expression is decreased.

15. The method of claim 9 wherein the composition is in a form suitable for topical application and preferably in the form of a liquid, a lotion, an aerosol, a cream, a gel or an ointment.
